(19) United States
(12) Patent Application Publication
(10) Pub. No.: US 2005/0208087 A1
(43) Pub. Date: Sep. 22, 2005

(54) Modified oxidic nano-particle with hydrophobic inclusions, method for the production and use of said particle

(76) Inventors: Konrad Kuerzinger, Penzberg (DE); Dragan Griebel, Wurzburg (DE); Albert Geiger, Penzberg (DE); Rupert Hermann, Weilheim (DE)

Correspondence Address: Roche Diagnostics Corporation, 9115 Hague Road, PO Box 50457, Indianapolis, IN, 46250-0457, US

(21) Appl. No.: 10/506,862

(22) PCT Filed: Mar. 4, 2003
(86) PCT No.: PCT/EP03/02198

§ 371 Date: May 9, 2005

(30) Foreign Application Priority Data
Mar. 6, 2002 (DE) 102 09 744.5

(51) Int. Cl. C01F 1/00; C07F 9/00; A61K 7/42; A01N 25/00; G03G 9/00

(52) U.S. Cl. 424/405; 423/265; 424/59; 436/523; 430/108.6; 556/42

(57) ABSTRACT

The invention concerns processes for producing modified oxidic nanoparticles with hydrophobic inclusions, in particular metal oxide particles which contain halogen-containing target molecules; the particles produced in this manner and the use thereof especially as a toner, sunscreen agent, insecticide or for labelling biomolecules.

The invention concerns processes for the production of modified metal-oxidic nano-particles with hydrophobic inclusions, in particular metal oxide particles which contain halogen-containing target molecules; the particles produced in this manner and the use thereof especially as a toner, sunscreen agent, insecticide or for labelling biomolecules.

Latex particles are hydrophobic and are very suitable as a host for hydrophobic molecules and used as such (Kawaguchi, H., Prog. Polym. Sci. 25 (2000) 1171-1210). Although organic nanoparticles dispersed in water are being used increasingly in pharmaceuticals, cosmetics, plant protection and foods, solvent residues are for example still present which can have an adverse effect on the respective applications. These problems are at present being intensively researched (Horn, D., and Rieger, J., Angew. Chem. 113 (2001) 4460-4492).

Since the metal oxide particles are synthesized by wet chemical methods in a water-ethanol mixture they naturally contain no interfering surfactants, stabilizers etc. In addition a multifunctional surface is present which can be modified depending on the requirements, for example with carboxyl functionalities (as biolinkers) or fluoroorganyl groups (to influence the physicochemical surface properties). Metal oxide particles are by nature hydrophilic and are therefore unsuitable as a host for hydrophobic molecules.

Nanoparticles based on silicate which are stained with hydrophilic dyes have been known for a long time in the prior art. They are used in the form of pigments for example as dyes for toners and inks, for plastic materials and also as labelling and carrier materials in the medical engineering field.

On an industrial scale silicate particles are usually produced by flame hydrolysis (e.g. Aerosil®). Silicate particles obtained in this manner can be coloured on their surface or in layers.

U.S. Pat. No. 5,102,763 describes the use of hydrophilic, coloured SiO₂ particles for use as toners. The surface of these particles is covalently stained by reacting pre-activated silicate particles with various dyes.

The production of coloured particles by covalently binding a dye to the surface of particles is described in WO 93/10190.

Silicate particles which are only coloured on the surface have a tendency to lose colour by bleeding. This results in a reduction in the colour intensity and these particles are also often no longer uniformly coloured. The use of these particles to produce conjugates that are suitable for diagnostic agents is not described.

A process for producing coloured particles with a silicate surface is described in the U.S. Pat. No. 5,209,998. The production process is based on the coating of coloured pigments with a silicate shell. Hence only the nucleus of these particles is coloured. The use of the particles in electrostatic toners, plastic materials and inks is described as the application; a diagnostic application is not disclosed.

A process for producing monodisperse silicate particles i.e. silicate particles of a uniform size, is the sol-gel process. It was first described by Stöber et al., (Colloid J. Interface Sci. 26 (1968) 62-69). The production of so-called Stöber particles and their properties were subsequently extensively examined by numerous groups. These studies encompassed the determination of the synthesis conditions required to obtain certain particle sizes (Van Helden, et al., Colloid J. Interface Sci. 81 (1981) 354-68; Giesche, H., J. European Ceramic Soc. 14 (1994) 189-204, Van Blaaderen, A., and Vrij, A., Adv. Chem. Ser. 234 (1994) 83-111) as well as investigations on particle growth and chemical composition (Byers, C. H., et al., Ind. Eng. Chem. Res. 26 (1987) 1916-1923; Matsoukas, T., and Goulari, E., Colloid J. Interface Sci. 124 (1988) 252-261; Harris, T., et al., J. Non-Cryst. Solids 121 (1990) 307-403; Matsoukas, T., and Gulari, E., Colloid J. Interface Sci. 132 (1989) 13-21; Badley, R. D., et al., Langmuir 6 (1990) 792-801).

Various methods have been described in the prior art for doping silicate particles from the sol-gel process with dyes.

Van Blaadereno, et al., Langmuir 8 (1992) 2921-2931 and Quellet, et al., Colloid J. Interf. Sci. 159 (1993) 150-7 produced Stöber particles that were stained with fluorescein isothiocyanate or rhodamine isothiocyanate (Verhaegh and Van Blaaderen, A., Langmuir 10 (1994) 1427-1438). The dyes were previously reacted with 3-aminopropyltriethoxysilane (AMEO). In this case the dye was covalently attached to the surface or covalently incorporated into the particles in layers. The resulting inhomogeneous staining was of secondary importance in these investigations and the method usually resulted in relatively large particles in a size range of about 500 nm diameter. The particles obtained were used as model systems for basic research. The large particle size makes silicate particles produced in this manner less suitable for diagnostic applications.

Shibata, S., et al., J. Sol-Gel Sci. And Techn. 10 (1997) 263-268 physically doped Stöber particles with various hydrophilic dyes such as rhodamine 6G, water-soluble porphyrins, Nile-blue etc. Schwert, R., Dissertation Würzburg 2000 found that only cationic but not anionic or hydrophobic dyes can be incorporated physically (non-covalently) in the Stöber process.

Matijevic, et al., Dyes and Pigments 17 (1991) 323-340 presented Stöber particles whose surface was modified with 3-aminopropyltriethoxysilanes which were linked via the amino group with dyes in a complicated process. The surface of Stöber particles was also modified in various other manners. These include reactions with 3-methacryloxypropyltrimethoxysilane (MEMO), octadecyltrimethoxysilane (ODS) and 3-aminopropyltriethoxysilane (AMEO) (Giesche, H., and Matijevic, E., Dyes and Pigments 17 (1991) 323-340; Van Blaaderen, A., and Vrij, A., Golloid J. Interface Sci. 156 (1993) 1-18; Badley, R. D., et al., Langmuir 6 (1990) 792-801; Philipse, A. P., and Vrij, A., Colloid J. Interface Sci. 12 (1989) 121-136; Van Helden, A. K., and Vrij, A., Colloid J. Interface Sci. 81 (1981) 354-368).

Homogeneously coloured silicate particles can be produced in the sol-gel process by covalent dye incorporation (EP 1 036 763). However, the dyes have to be firstly silanized before they can be used in this process. A covalent incorporation is only possible in this manner.

Many important target molecules for incorporation into nanoparticles and in particular many dyes carry halogen groups as substituents. These dyes are not only hydrophobic but also oleophobic.

Fluorine-containing coatings based on SiO₂ are known (Lotus-Effect, Easy to clean surfaces, adjustment of refractive numbers—Kron J., et al., 2^{nd} Wörlitzer Workshop: Functional layers—adhesive and antiadhesive surfaces (“Fördergemeinschaft Dünne Schichten e.V.”), Conference paper 2000. However, due to the rapid gelling during the particle production with fluoroalkyltrialkoxysilanes, no fluorine-containing silicate particles have yet been synthesized. But these would be desirable in order to also enclose hydrophobic and especially oleophobic molecules in SiO₂ particles.

Hence the object of the invention was to modify the production process for metal oxide particles in such a manner that hydrophobic complexes or hydrophobic organic dyes can for example be integrated into SiO₂ particles.

Hence the object was to develop a process which enables the incorporation of hydrophobic and in particular oleophobic dyes into metal oxide particles.

The object is achieved by the invention which is defined in more detail in the independent claims. The dependent claims represent preferred embodiments.

It was surprisingly found that it is possible to produce metal oxide particles in the sol-gel process in the presence of fluoroorganylalkoxysilane or arylalkoxysilane and to non-covalently incorporate hydrophobic and in particular oleophobic target molecules into these nanoparticles in this production process.

The invention concerns sol-gel processes for producing a metal oxide particle which contains at least one target molecule containing halogen in which, starting from known metal oxide precursors, the said precursor and the said target molecule are used, characterized in that a polyhalogenated metal alkyl-alkoxy compound, in particular alkylalkoxysilane is additionally used in the said sol-gel process.

A sol-gel process is understood as any process which can be used in analogy to the process described by Stöber et al. (1968), supra to produce colloidal nanoparticles. The products of this process are referred to as Stöber particles or nanoparticles.

The invention concerns the non-covalent incorporation of halogen-containing target molecules into metal oxide particles. The target molecules in the sense of this invention consist of 5-65 percent by weight (=weight %) halogen and preferably have a molecular weight of between 250 and 5000 Dalton. Target molecules are in particular halogen-containing dyes and halogen-containing insecticides.

The halogen-containing target molecule is not silanized. Hence their incorporation into the Stöber particles is non-covalent.

The process according to the invention is especially characterized in that for the first time it has been possible to produce Stöber particles in the presence of a polyhalogenated metal alkylalkoxy compound. The process can be carried out in the presence or absence of a target molecule. A polyhalogenated metal alkylalkoxy compound contains a linear or branched alkyl residue with 2 to 20 carbon atoms which carries at least two halogen groups. The polyhalogenated alkyl residue preferably contains less than 30 halogen groups. Particularly preferred polyhalogenated metal alkylalkoxy compounds contain alkyl residues with 3 to 20 carbon atoms and 2 to 15 halogen groups. Metal alkylalkoxy compounds based on silicon, titanium or zirconium and in particular the alkylalkoxysilane are particularly preferred.

Metal alkoxides or metal halogenides are usually used as metal oxide precursors. Preferred metal alkoxides are silicon metal oxides in particular tetraethoxysilane (TEOS) and tetramethoxysilane (TMES).

In the original Stöber process using SiO₂ as the metal oxide, the SiO₂ particles are produced by hydrolysis and condensation of a silicon alkoxide which is usually tetraethoxysilane (TEOS). The reaction takes place in a mixture of water, ammonia and a lower alcohol, usual ethanol. The main reactions in the formation of the SiO₂ particles can be described as follows:

1) Hydrolysis
≡Si—OR+H₂O→≡Si—OH+ROH

2) Condensation I
≡Si—OR+=≡Si—OH→≡Si—O—Si≡+ROH

3) Condensation II
≡Si—OH+≡Si—OH→≡Si—O—Si≡+H₂O

4) Total reaction
Si(OR)₄+2H₂O→SiO₂+4 ROH

In the synthesis alcohol, water and ammonia are added first and subsequently TEOS is added. Depending on the synthesis conditions, the solution becomes opalescent after a few seconds to minutes. This induction period increases with decreasing particle size and temperature. The size of the particles obtained has a standard deviation of 2-8%. During the reaction the alcohol serves as a cosolvent for the water-insoluble TEOS. The ammonia catalyses the hydrolysis as well as the condensation reaction. The base deprotonates the surface silanol groups of the formed particles. The resulting negative charges stabilize the colloidal system as a result of electrostatic repulsion. Hence the suspensions remain stable for several months to years. At the same time the silanol groups that are present enable a functionalization of the particle surface (various examples thereof have already been described in the literature e.g. AMEO, ETEO, MEMO, MPTMO, GLYMO, GF20. Their dispersibility in various solvents can be varied by suitable surface modification.

The particle size can be controlled by the ammonia and water concentration, the reaction temperature and the solvent. The following trends are seen:

1) Increasing the ammonia as well as the water concentration accelerates the reaction and increases the particle size.

2) The particle size increases and the monodispersity decreases with increasing chain length and branching of the alcohol.

3) The particle size decreases with an increasing length of the alkoxide residues of the silane. The ionic strength of the reaction solution (salt effect) can also influence the particle size due to compression of the electrostatic double layer.

4) If the TEOS concentration exceeds 0.2 mol/l, the particles become more polydisperse and less spherical.

The previously discussed influences on particle formation in the original Stöber process apply analogously to a process according to the invention in which a polyhalogenated metal alkylalkoxy compound is additionally used in order to for example incorporate a halogen-containing target molecule in the Stöber particles obtained by this process.

The process according to the invention can for example be carried out by simultaneously reacting the metal oxide precursor, halogen-containing dye and polyhalogenated metal alkylalkoxy compound components under suitable reaction conditions known to a person skilled in the art.

The halogen-containing target molecule and the polyhalogenated metal alkylalkoxy compound are preferably dissolved in advance in a suitable solvent, mixed and added together.

A sol-gel process comprising the following steps is particularly preferred for producing a metal oxide particle containing at least one halogen-containing target molecule a) production of a mixture containing the target molecule and a polyhalogenated metal alkylalkoxy compound, b) starting the sol-gel process with a metal oxide precursor, c) adding the solution from a), d) optionally further addition of the metal oxide precursor and e) ending the sol-gel process.

The quantity ratios of the metal oxide precursor that are used in the above steps b) and d) can vary over a wide range. Preferably between 90 to 10% of the total amount of metal oxide precursor used in the process is used in step b) and correspondingly the remaining 10 to 90% is used in step d). The partial amount used in step b) is particularly preferably 75 to 25% and in step d) 25 to 75%.

Also the time period for starting the sol-gel process in step b) is variable. It is preferably less than 1 h, more preferably between 1 and 20 min and particularly preferably between 2 and 10 min.

It has proven to be particularly suitable to coordinate the molar ratios of metal oxide precursor and polyhalogenated metal alkylalkoxy compound. Preferably 0.04 to 0.4 mol % polyhalogenated metal alkylalkoxy compound, particularly preferably 0.1 to 0.3 mol % based on the metal oxide precursor are used.

The halogen-containing target molecules preferably contain between 10 and 65 weight, particularly preferably between 15 and 50 weight % halogen and the molecular weight is preferably between 250 and 5000 Dalton, more preferably between 300 and 4000 Dalton and especially preferably between 400 and 3000 Dalton.

Preferred halogens in the halogen-containing target molecules are fluorine and chlorine.

The amount of added target molecule can vary according to needs. Of course it is also possible to prepare particles which contain no target molecules or only minimal amounts thereof. Preferably between 0.1 and 10% by weight target molecule and particularly preferably between 0.2 and 5 weight % based on the metal oxide precursor is used.

Oxides of the elements from groups III, IV and IVb of the periodic system come into special consideration as metal oxides or as components of mixed oxides. The metal oxide precursor is preferably selected such that in addition to the inclusions of the target molecule and the covalently incorporated polyhalogenated metal alkyl, the Stöber particles are essentially composed of B₂O₃, Al₂O₃, SiO₂, SnO₂, ZrO₂ or TiO₂.

Of course particles based on mixed oxides can be used in an analogous manner in the inventive sol-gel process.

Metal oxide precursors based on boron, silicon or zirconium are particularly preferably used, silicon precursors being especially preferred.

The present invention also concerns the particles that can be obtained by the process according to the invention.

These are in particular particles which were obtained by hydrolysis and condensation of sol-gel precursors of elements of groups III, IV, IVb, preferably Si (Ti, Zr, Al) in combination with hydrophobic sol-gel precursors such as perfluorinated alkyltrialkoxysilanes (e.g. 3,3,3-trifluoropropyltrimethoxysilane) or bis(trialkoxysilylalkyl)benzenes (e.g. bis(trimethoxysilylethyl)benzene). These particles preferably contain the above-mentioned target molecules.

Due to the hydrophobic (fluorinated) environment that is present in the case of the metal oxide particles produced according to the invention, fluorophores for example do not exhibit the otherwise common adverse effects of water i.e. quenching due to water does not occur.

The modification of the particle interior according to the invention enables other hydrophobic (e.g. LC Red 640) as well as oleophobic molecule/complexes to be incorporated into the originally highly polar oxidic matrix in addition to lanthanoid complexes. The particle surface can be functionalized as required for example with carboxyl, amino, mercapto, epoxy and aldehyde groups. This can be accomplished among others by silanization. The particle size can be adjusted from the nano- to micrometer range with a narrow size distribution.

The particle type (cf. FIG. 1) is not decisive. The particles are preferably composed of an inorganic-oxidic core. This core can have a homogeneous (type 1) or heterogeneous (core-shell type (2) or currant cake model (3)) composition.

The Stöber particles according to the invention loaded with a halogen-containing target molecule can be very advantageously used in various technical fields. They are especially suitable as labels for biomolecules and hence for applications of the labelled biomolecules in immunological and other detection methods, as toners in the printing industry, as sunscreen agents and as insecticides. It is also possible to incorporate them into any polymer matrix (e.g. Ormocer®). The applications as labels for biomolecules or as an insecticide are particularly preferred.

The metal oxide particles according to the invention can be subsequently modified. Thus the particles can for example be coated with one or more additional, preferably colourless layers in order to chemically protect the particles. The purpose of this coating is to obtain a metal oxide surface e.g. a silicate-like surface that is as uniform as possible from which colour molecules no longer protrude. This facilitates additional coupling with functional groups and biomolecules and reduces the risk of secondary reactions with dye molecules on the surface. Preferably an additional uncoloured silicate layer is applied at a thickness of 1 to 30 nm, preferably 2 to 20 nm to the homogeneously coloured silicate particles.

The metal oxide particles according to the invention can either be provided directly with functional groups or they can be provided on the surface of the additional coating layer in order to couple additional molecules to the particle which according to the invention are preferably biomolecules.

The functional groups can in turn be attached to the particles via spacer or linker molecules. It is important that the functional group to be introduced is anchored in the network of the metal oxide particle in order to ensure a stable linkage.

Preferred modification groups are functional groups such as carboxyl groups, amino groups, epoxy groups, hydroxyl groups or thiol groups. A person skilled in the art knows how to introduce such groups. It does not therefore have to be separately elucidated.

It is preferable to introduce carboxyl groups which is preferably carried out by reacting the coloured metal oxide particles with a dye acid anhydride which contains the said silanol group for anchoring in the particle. In order to activate the functional groups they can for example be converted into active esters with N-hydroxy-succinimide before reaction with the biomolecules to be coupled. All these steps are familiar to a person skilled in the art.

The conjugates according to the invention are composed of metal oxide particles loaded with a halogen-containing dye and biomolecules. The biomolecules are preferably coupled via the functional groups that are introduced on the surface. In general the biomolecules are linked to the surface of the particle via free amino or carboxyl groups or thiol groups such that the covalent linkage is preferably via amide or thioether bonds.

Biomolecules in the sense of the present invention are understood as all molecules that can be used to determine an analyte in a sample, in particular for an immunological determination of an analyte. The term biomolecule for example includes proteins, glycoproteins, peptides, nucleic acids, peptidic nucleic acids, saccharides, hormones, haptens, vitamins, naturally occurring or artificially produced binding partners and antigens. Antibodies and fragments thereof are preferably used as biomolecules in the conjugate according to the invention. Antibodies are understood to include monoclonal as well as polyclonal antibodies and chimeric antibodies and fragments thereof such as Fab, Fc, Fab′, F(ab′)₂, Fv, scfv. Coupling to the biomolecules streptavidin or avidin or biotin is also one of the preferred embodiments of the invention.

Conjugates of the inventive metal oxide particles and biomolecules are a further subject matter of the invention. These inventive conjugates are preferably used in a method for detecting an analyte in a sample by contacting the sample with one or more analyte-specific binding partners.

The method for detecting an analyte is preferably carried out as an immunoassay. This means that at least one of the analyte-specific binding partners is an immunological binding partner. In this method the sample which is presumed to contain the analyte is incubated with an immunologically specific binding partner. In the case of an antigen test for example for tumour markers such as PSA, an antibody or a fragment thereof which specifically binds to the analyte, i.e. the tumour antigen PSA, is the said immunologically specific binding partner. In methods for detecting antibodies to a certain antigen (e.g. anti-HCV antibodies) the corresponding antigen can for example be used as the immunologically specific binding partner. The specific binding is detected by means of the inventive conjugate whose incorporated dye serves as a label. The biomolecules immobilized on the metal oxide particles act as specific binding partners for the analyte or as specific binding partners for a substance which in turn is specifically bound to the analyte.

For example in a diagnostic test procedure, streptavidin or avidin can be conjugated as the biomolecule to the metal oxide particle. The conjugate then binds to the biotin group of a molecule (for example a peptide antigen or a nucleic acid sequence) that is itself biotinylated.

Immunoassay procedures and nucleic acid test procedures are familiar to a person skilled in the art.

The conjugates according to the invention are preferably used in a test based on a test strip. The following describes, as an example, how a test strip is constructed and how such a test procedure is carried out.

Test strips are usually composed of a carrier material on which an application fleece, a membrane and a suction fleece are mounted. The conjugate according to the invention whose biomolecules are specific for the analyte and optionally other specific binding partners for the analyte are applied and dried upstream of the chromatography direction i.e. above the starting point for the sample liquid. The specific binding partners and the inventive conjugate do not begin to migrate chromatographically until contact with a liquid i.e. with the sample. Various proteins are also applied to the membrane in the direction of chromatography in the form of two successive strips or lines.

An immobilized binding partner that is specific for the analyte is located on the first line (result line). A molecule such as streptavidin can also be bound to the first line to which biotinylated, analyte-specific binding partners can then bind. In this case the biotinylated, analyte-specific binding partners as well as the conjugate must be applied above the starting point of the test strip and chromatographed together with the sample. A binding partner which specifically binds the biomolecules of the inventive conjugate is applied to the second line in the direction of chromatography (control line).

As the sample liquid migrates from the starting point of the test strip through the strip, the conjugate according to the invention and optionally the analyte-specific binding partner also begin to migrate towards the liquid front. In this process the analyte from the sample specifically binds to the binding partners immobilized on the first line. The inventive conjugate also binds to the analyte to form a sandwich that can be detected by means of the colour of the metal oxide particles. The liquid in the test strip runs further up to the end of the test strip. In this process the inventive conjugate that is not consumed by analyte binding is captured on the second line by the binding partner that specifically binds the biomolecules of the conjugate. One can see on the basis of the colouration of the control line that the chromatography in the test strip has basically worked and/or is completed.

Another subject matter of the invention is a diagnostic test strip which, in addition to the conjugate according to the invention, contains all other components necessary to carry out the chromatographic test.

According to the invention the conjugates can also be used in nucleic acid hybridization assays. In this case a nucleic acid probe which specifically hybridizes with a nucleic acid sequence to be detected is coupled as a biomolecule with the metal oxide particles that are coloured according to the invention. The nucleic acid sequence from the sample or from a mixture that is for example obtained by PCR amplification can be specifically detected by means of the dye contained in the metal oxide particles.

According to the invention the conjugates comprising the metal oxide particles according to the invention and a biomolecule can also be used in array or chip systems. Such systems are miniaturized test designs. Spatially separated reagent spots are applied with a very small spacing which is in the micrometer range to the surface of suitable solid phases such as plastics, glass, metals or metal oxides. These reagent spots contain the specific binding particles required to carry out the respective detection method. Such detection methods enable numerous different analytical parameters to be detected simultaneously and rapidly in a very small space using little material and sample. The conjugates according to the invention comprising metal oxide particles coloured with halogen-containing dyes and biomolecules can also be used as detection reagents in these array or chip systems. Suitable dyes for colouring the metal oxide particles are preferably fluorescent dyes and especially those that enable a time-resolved measurement of fluorescence. In particular the conjugates according to the invention enable differently coloured and/or different conjugates loaded with different biomolecules to be used in order to simultaneously detect different analytes by means of the different (fluorescent) dyes. Such array systems have proven to be particularly advantageous for nucleic acid hybridization assays.

The simultaneous detection of a plurality of different analytes (for example HIV- and HCV-specific nucleic acids in a sample or HIV- and HCV-specific antibodies in a sample) by means of the conjugates according to the invention which are each differently coloured and/or loaded with different biomolecules is not limited to an application in array systems but is particularly appropriate therefor.

All body fluids can be used as the sample material for all diagnostic test methods. Whole blood, serum, plasma, urine, sweat or saliva are preferably used.

Another subject matter of the invention is the use of the conjugates of metal oxide particles according to the invention and biomolecules in a diagnostic, preferably immunological method to detect an analyte in a sample.

A diagnostic reagent which contains conjugates according to the invention is also a subject matter of the invention. The reagent can additionally contain the buffer additives, salts or detergents known to a person skilled in the art.

A test kit which contains the conjugates according to the invention and other common reagents known to a person skilled in the art for carrying out a test is also one of the preferred embodiments of the present invention.

Many important insecticides have a high halogen content. These insecticides are preferred target molecules for the process according to the invention for non-covalent incorporation into metal oxide particles and in particular into silicate particles.

The digestive tract of insects and especially of insect larvae differs fundamentally from that of mammals. Whereas there is a strongly acidic pH the stomach of mammals, food is digested in the digestive tract of insect larvae in a strongly alkaline pH range.

Metal oxide particles especially those based on silicate or mixed oxide particles containing more than 20% silicate have the special property that they swell under alkaline pH conditions such as those that are for example present in the digestive tract of insects and release non-covalently incorporated components, in particular polyhalogenated insecticides. Since the insecticide target molecules are not covalently bound in the particles according to the invention, they are released and are effective in the insect intestine i.e. precisely at the intended site of action.

The inclusion of insecticide agents in metal oxide particles by the sol-gel process of the present invention additionally has the effect that the agents are for example protected from water. The insecticidal effect only occurs after intake of food by the insect. Sol-gel particles according to the invention containing insecticides with incorporated insecticidal agents are less poisonous and/or more environmentally friendly than the free agents.

Halogen-containing dyes having spectral properties that are important for the printing industry can be incorporated into sol-gel particles in the process according to the invention. Such particles are used especially as an admixture in so-called toners.

The halogen-containing substances which can be incorporated according to the invention into metal oxide particles include many substances which absorb and suppress damaging UV light or release it again as longer wavelength less damaging light. Metal oxide particles according to the invention which contain such substances are preferably used in the cosmetic industry especially as sunscreen agents.

The invention is further elucidated by the following examples, publications and figures the protective scope of which results from the claims. The described processes are to be understood as examples which still describe the subject matter of the invention even after modifications.

### DESCRIPTION OF THE FIGURES

FIG. 1: Schematic representation of homogeneous (type 1) or heterogeneous (core-shell (type2), currant cake (type 3) particles having an inorganic-oxidic matrix.

FIG. 2: Structural formula of Tris-[4,4,4-trifluoro-1-(2-naphthyl)-1,3-butanedione]-Eu(III) complex (Eu(NTA)3 complex).

FIG. 3: UV-Vis spectrum of the Eu(NTA)3 complex in CH₂Cl₂/EtOH (1:1) λ_{abs}.=333 nm.

FIG. 4: fluorescence spectrum of the EU(NTA)3 complex in CH₂Cl₂/EtOH (1:1) λ_{exc}.=333 nm.

Assignment of the Fluorescence Bands to the Spectral Transitions

| Emission bands | assignment* |
|---|---|
| λₑₘ = 578 nm | ⁵D₀ → ⁷F₀ |
| λₑₘ = 590 nm | ⁵D₀ → ⁷F₁ |
| λₑₘ = 612 nm | ⁵D₀ → ⁷F₂ |
| λₑₘ = 651 nm | ⁵D₀ → ⁷F₃ |
| λₑₘ = 699 nm | ⁵D₀ → ⁷F₄ |

*Lit.: R. Reisfeld et al., J. of alloys and Compounds 300-301 (2000), 147-151

FIG. 5: Measurement of a solid specimen of silicate particles containing the Eu(NTA)3 complex which were fixed on a microscope slide.

Settings:

power=950 mV, slit widths=EX/EM=10/1
light source=xenon lamp
λ_{exc}.=333 nm
λₑₘ.=613 nm

FIG. 6: IR spectrum of the silicate particles doped with the Eu(NTA)3 complex on a pressed piece of KBr

Assignment of the Bands

ν(O—H)=3430 cm⁻¹
δ(H₂O)=1640 cm⁻¹
ν(Si—O—Si)=1100 cm⁻¹ (as)
ν(Si—O—Si)=800 cm⁻¹ (sym)
δ(Si—O—Si)=471 cm⁻¹ (?)

Assignment according to: Fendler, J. H., Nanoparticles and nanostructured Films, Wiley-VCH 1998, 180-183.

FIG. 7: Raman spectrum of a solid specimen of silicate particles doped with an Eu(NTA)3 complex

Assignment of the Bands:

ν(C—H, aliph.)=2943, 2875 cm⁻¹ (as)
δ(CH₃, CH₂)=1452 cm⁻¹
ν(Si—O—Si)=1068 cm⁻¹ (as)
ν(Si—O—Si)=839, 793 cm⁻¹ (sym)
δ(Si—O—Si)=482 cm⁻¹

Assignment according to: J. H. Fendler, supra

FIG. 8: TEM pictures of 130-158 nm silicate particles doped with 4.9 μmol Eu(NTA)3 complex per g SiO₂ at 6300-fold (FIG. 8 left) and 63000-fold enlargement (FIG. 8 right)

FIG. 9: VACP/MAS 13C solid NMR spectrum of the Eu(NTA)3 complex

Interpretation:

129.4/126.7 ppm; arom. C—H
61.2 ppm; CH₂—OH
27.6 ppm; CH₂—CH₂—CF₃
17.4 ppm; CH₃—CH₂—OH
4.5 ppm; Si—CH₂—CH₂—CF₃

FIG. 10: MAS ²⁹Si solid NMR of silicate particles doped with the Eu(NTA)3 complex.

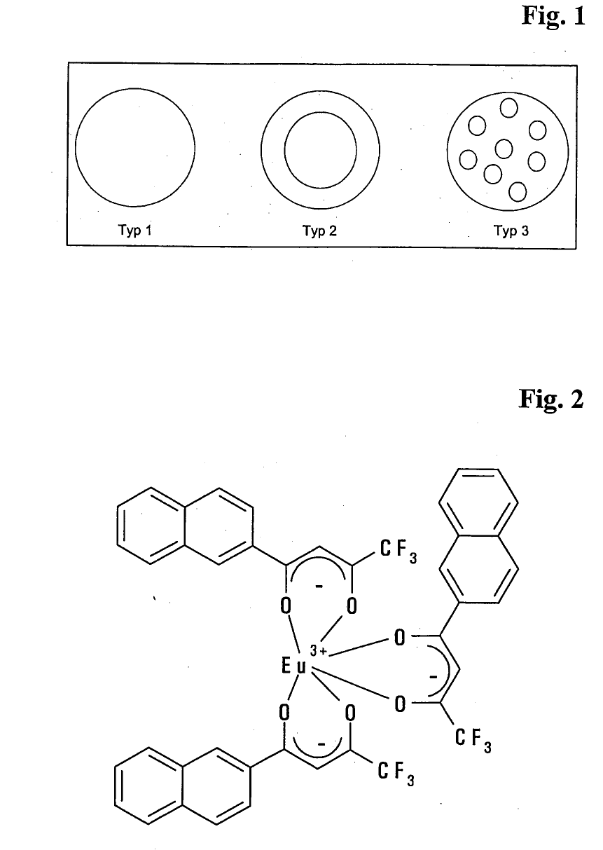

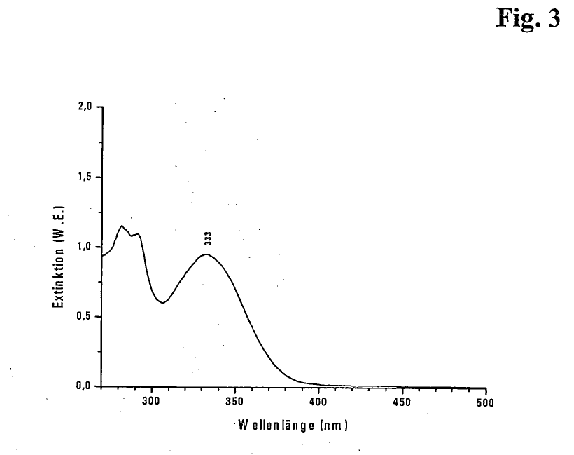

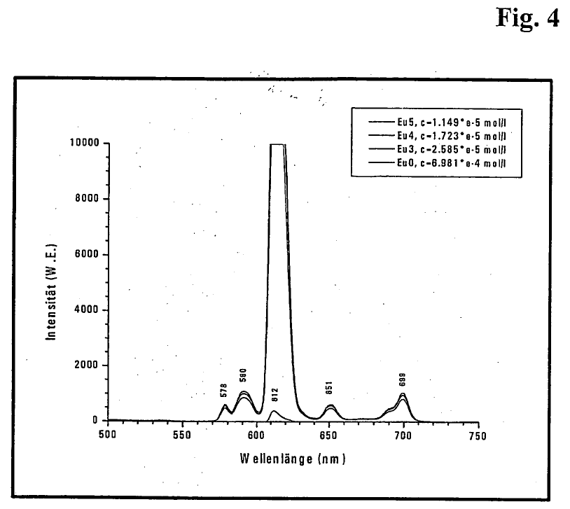

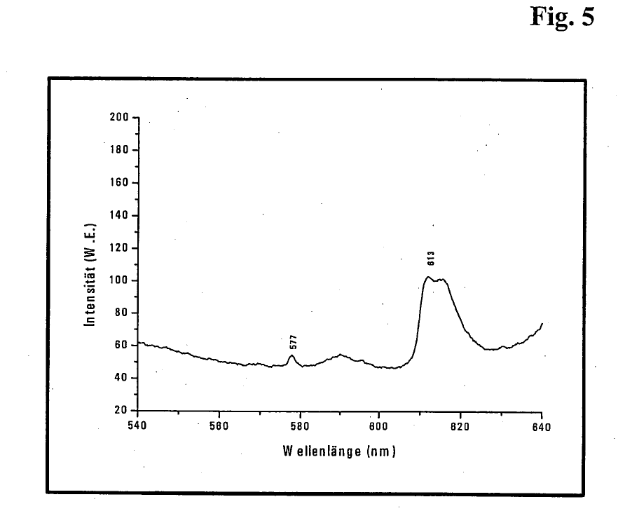

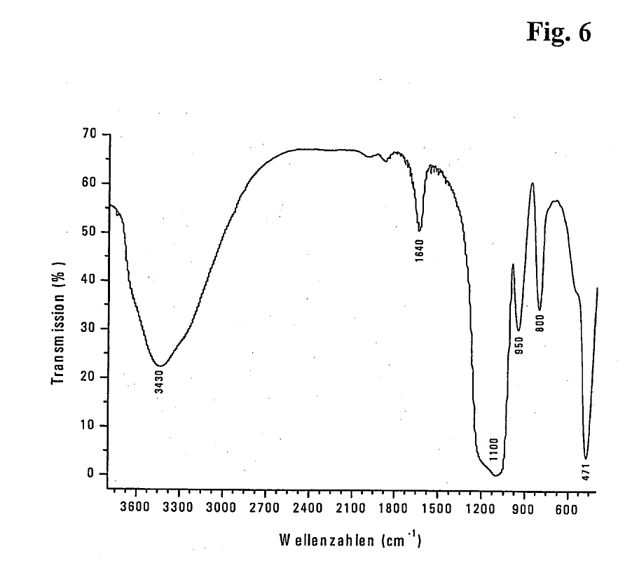

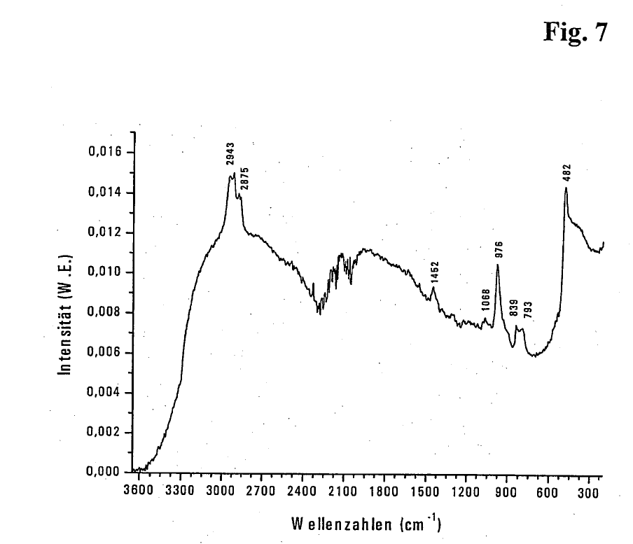

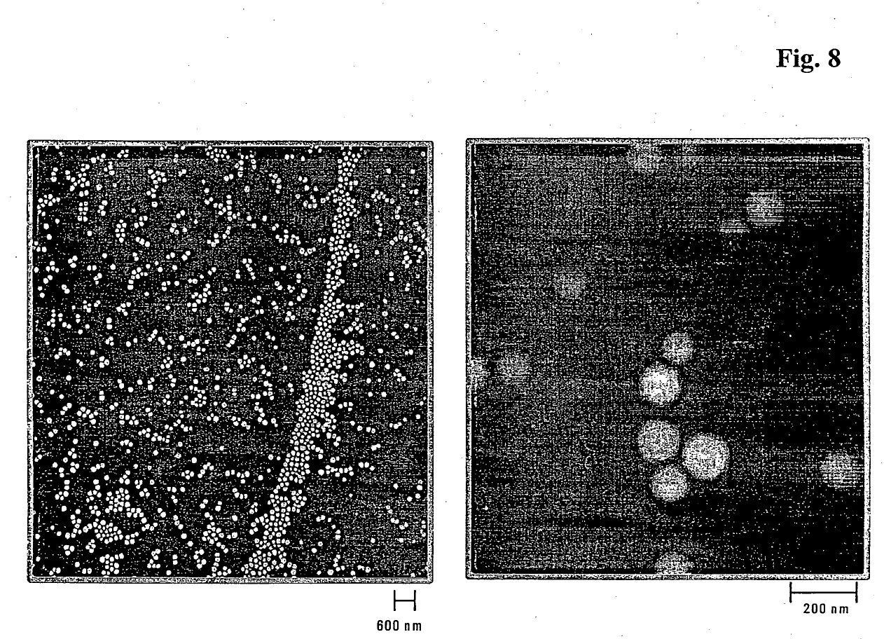

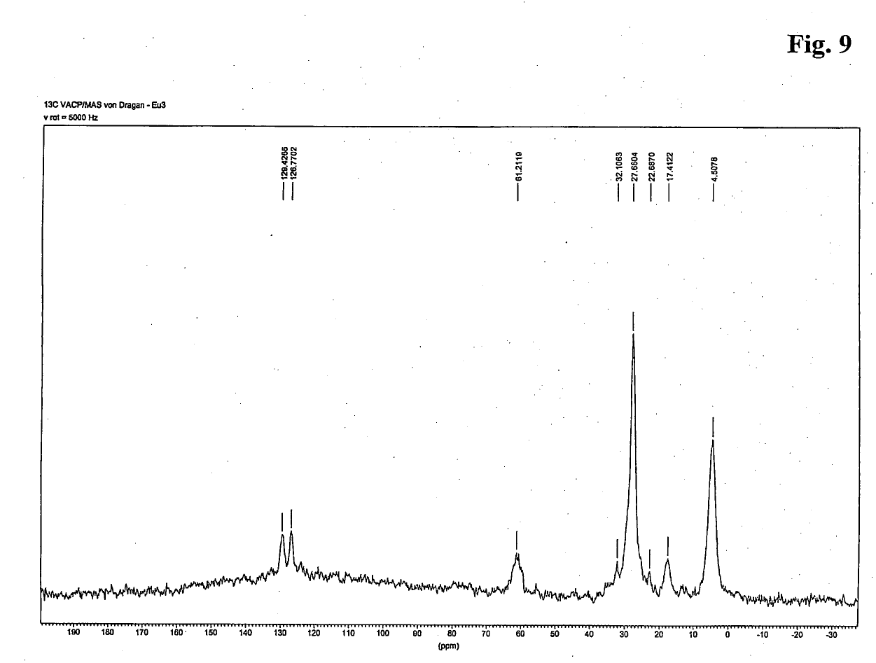

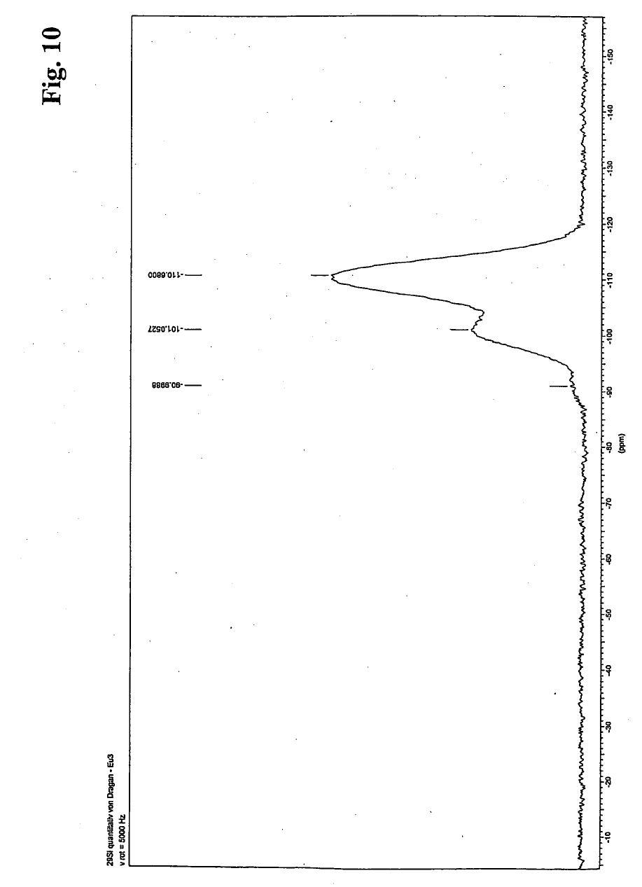

Integration of the Signals Yielded the Following Distribution:

110.7 ppm; Q4-groups, 70.54%
101.1 ppm: Q3-groups, 27.24%
91.0 ppm: Q2-groups, 2.21%

### ABBREVIATIONS USED

AMEO 3-aminopropyltriethoxysilane
<Dig> anti-digoxigenin
ETEO ethyltriethoxysilane
GF20 2(3-triethoxysilylpropyl)-succinic anhydride
GLYMO glycidoxypropyltrimethoxysilane
Ig immunoglobulin
LCR LightCycler Red
MAB monoclonal antibody
MEMO methacryloxypropyltrimethoxysilane
MES 2(N-morpholino)ethanesulfonic acid
MPTMO 3-mercaptopropyltrimethoxysilane
BPLA bovine plasma albumin
SA streptavidin
Si—NP silicate nanoparticle
TEOS tetraethoxysilane
TMES tetramethoxysilane

### EXAMPLE 1

### General protocol for preparing lanthanide (III)-tris-4,4,4-trifluoro-(1-naphthoyl)-1,3-butanedione complexes

800 mg (3 mmol) 4,4,4-trifluoro-1-(2-naphthoyl)-1,3-butanedione was dissolved in 15 ml ethanol. Subsequently 3 ml of a 1 M NaOH solution was added to this solution. 1 mmol lanthanum (III) chloride or lanthanum (III) nitrate was dissolved in 5 ml water in a dropping funnel and then slowly added dropwise to the reaction solution. Afterwards a further 100 ml water was added to the reaction mixture and it was stirred for 1 hour at 65° C. The product was filtered off as a pale yellow solid and washed three times with 5 ml water and ethanol each time. It was finally dried for 3 hours at 120° C. in a drying cabinet.

This general procedure was used to prepare terbium (III), gadolinium (III), dysprosium (III), and erbium (III) complexes.

### EXAMPLE 2

### Review of Physical Dye Incorporation into Fluorine-Free (“Normal”) Silicate Nanoparticles (Si—NP) and Organofluorine-Modified (Fluorinated) Si—NP

### 2.1 Preparation of Normal Silicate Particles Coloured with LightCycler Red 640™ (Reference Particles)

41 mg (4.29*10⁻⁵ mol) LightCycler Red 640™ LCR 640 was dissolved in 330 ml 99% ethanol. 168 ml demineralized water and 11 ml of a 14 molar ammonium hydroxide solution were added to this solution. The solution was heated to 35° C. After a thermal equilibrium was established, 24 ml (107 mmol) tetraethoxysilane (TEOS) was added while stirring vigorously. The reaction was fully completed after 24 h. A coloured dispersion having a solids content of about 2% by weight was obtained. The particles have a size of about 135 nm diameter. These particles were purified of non-incorporated dye by centrifuging and redispersing three times in fresh ethanol.

### 2.2 Preparation of Fluorinated Silicate Particles Coloured with LightCycler Red 640™

a) Protocol for Particles Containing 0.3% Fluoroalkylsilane

23.8 μmol LightCycler Red 640™, then 6 ml TEOS and 30 μl (155 μmol) 3,3,3-trifluoropropyltrimethoxysilane (ratio of LCR 640™: fluoroalkylsilane=1:7) were added to a solution comprising 165 ml EtOH, 84 ml H₂O and 5.5 ml NH₄OH heated to 35° C. After stirring for 5 minutes the remaining 6 ml TEOS was added to the reaction mixture. The reaction was terminated after 8 h and the particles were separated by centrifugation. The particles were redispersed in H₂O and purified by centrifuging and redispersing several times.

b) Protocol for Particles Containing 0.2% Fluoroalkylsilane

23.3 μmol LightCycler Red 640™, then 5 ml TEOS and 20 μL (119 μmol) 3,3,3-trifluoropropyltrimethoxysilane (ratio of LCR 640™: fluoroalkylsilane=1:5) were added to a solution comprising 31 ml EtOH, 20 ml H₂O and 7 ml NH₄OH heated to 30° C. After stirring for 5 minutes the remaining 5 ml TEOS was added to the reaction mixture. The reaction was terminated after 8 h and the particles were separated by centrifugation. The particles were redispersed in H₂O and purified by centrifuging and redispersing several times.

### 2.3 Preparation of Silicate Particles Doped with Eu(III)-tris-4,4,4-trifluoro-1-(2-naphthyl)-1,3-butanedione

The Eu(III)-tris-4,4,4-trifluoro-1-(2-naphthyl)-1,3-butanedione complex was prepared according to the instructions of Charles, R. G., and Roedel, E. P., J. Inorg. Nucl. Chem. 29 (1967) 715-723.

a) Simultaneous Addition of Alkoxide and a Mixture of Polyhalogenated Alkylalkoxysilane and Halogen-Containing Target Molecule

20 ml TEOS and a mixture of 20 mg tris-[4,4,4-trifluoro-1-(2-naphthyl)-1,3-butane-dione]-Eu(III), 1 ml dichloromethane and 0.25 ml 3,3,3-trifluoropropyltrimethoxysilane were added to a solution comprising 61 ml water, 40 ml ethanol and 14 ml ammonium hydroxide solution heated to 30° C. The reaction mixture was stirred for 4 hours at 30° C. and for a further 10 h at room temperature. The particles were purified by centrifugation and firstly redispersed in ethanol and then in water in a subsequent washing step.
Incorporation rate (complex): 4.9 μmol/g SiO2
Calculated Eu content: 0.07%
Eu content found by X-ray fluorescence analysis (RFA): 0.05%
Particle size from TEM: 130-158 nm
   b) Successive Addition of Alkoxide and a Mixture of Polyhalogenated Alkylalkoxysilane and Halogen-Containing Target Molecule

61 ml water and 40 ml ethanol were heated to 30° C. in a 250 ml round bottomed flask. Then 14 ml ammonia solution and 10 ml TEOS were added. In parallel 19.4 mg (20.4 μmol) tris-[4,4,4-trifluoro-1-(2-naphthyl)-1,3-butanedione]-Eu(III) was dissolved in 1 ml dichloromethane, and 250 μl trifluoropropyltrimethoxysilane was added to the solution in an ultrasonic bath. After 5 minutes the solution was added dropwise to the preparation and stirred for a further 5 minutes. Subsequently another 10 ml TEOS was added and the reaction mixture was stirred for 4 hours at 30° C. and for a further 10 hours at room temperature. It was purified in several washing cycles using ethanol and water.

### 2.4 Preparation of the erbium (III) tris-(2,2′-bipyridyl)trichloride complex

1.1 g (7 mmol) 2,2′ bipyridine and 250 mg (0.7 mmol) erbium (III) nitrate (as an undefined hydrate complex) were added to 60 ml methanol. The reaction mixture was heated for 2 h to 60° C. while stirring vigorously. The complex precipitated as a yellow powder on cooling.

Incorporation into silicate particles was carried out as described in example 2.3a.

### 2.5 Preparation of the terbium (III) tris-(2,2′-bipyridyl)trichloride complex

1.47 g (9.43 mmol) 2,2′ bipyridine and 250 mg (9.43*10⁻⁴ mol) terbium (III) chloride hexahydrate complex were added to 60 ml methanol. The reaction mixture was heated for 2 h to 60° C. while stirring vigorously. The complex precipitated as a yellow powder on cooling.

Incorporation into silicate particles was carried out as described in example 2.3a.

### 2.6 Preparation of the terbium (III) tris-(1,10-phenanthroline)trichloride complex

482 mg (2.67 mmol) 1,10-phenanthroline and 250 mg (0.94 mmol) terbium (III) chloride hexahydrate were added to 20 ml methanol. The solution was stirred for 2 h at 60° C. and subsequently slowly cooled to room temperature (overnight). The yellow solution obtained in this manner was overlayered with n-pentane. The complex precipitates as a powder.

Incorporation into silicate particles was carried out as described in example 2.3a.

### 2.7 Summary of the Incorporation Behaviour of Various Dyes into Unmodified and Halogen-Modified Silicate Particles

incorporation into

| Incorporation of | Normal Si—NP | fluorinated Si—NP |
|---|---|---|
| fluorine-free dyes | ⊖ Tb(III)-bipy | ⊖ Tb(III)-bipy |
| | ⊖ Er(III)-bipy | ⊖ Tb(III)-phen |
| | ⊖ Tb(III)-phen | |
| Fluorinated or halo- | ⊖ Eu(NTA)₃ | ⊕ Eu(NTA)₃ |
| genated dyes | ⊖ LCR 640 | ⊕ Er(NTA)₃ |
| | | ⊕ LCR 640 |

Legend:
⊖ incorporation negative,
⊕ incorporation positive
bipy = α,α′-bispyridine
phen = 1,10-phenanthroline

### EXAMPLE 3

### Surface Modification with GF20 (Protocol for Introducing Carboxyl Groups)

The dispersion obtained in example 2.1 and 2.2 should not exceed a pH of 9.0. If necessary additional washing cycles have to be carried out (centrifugation/redispersion). 210 μl (75.4*10⁻⁵ mol) 2-(3-triethoxysilylpropyl)-succinic anhydride (GF20) was added to the resulting ethanolic dispersion in a volume of 250 ml while stirring vigorously. The reaction solution was stirred for 15 h at 40° C. The particles were purified by centrifugation and redispersion in water. This purification step was repeated a further two times. An aqueous dispersion of surface-modified particles is obtained with a coverage density of ca. 2 CO₂H groups/nm² particle surface.

### EXAMPLE 4

### Preparation of Conjugates of Silicate Particles Coloured with LightCycler Red 640 and Anti-Digoxigenin Antibodies (<Dig> Conjugates)

10 mg silicate particles (0.5 ml 2% suspension) was centrifuged for 30 min at 15000 rpm. The supernatant was removed and the pellet was resuspended in 1 ml 2 mM MES buffer pH 6.5. This washing process was repeated once more. Subsequently 100 μl 100 mM MES buffer pH 6.5, 100 μl 2% (w/v) sulfo-N-hydroxy-succinimide (S—NHS; Pierce No. 24510) in MES buffer pH 6.5 and 100 μl 0.2% (w/v) 1-ethyl-3-(3-diaminopropyl)-carbodiimide hydrochloride (EDC; Pierce No. 22980ZZ) in MES buffer pH 6.5 was added. After 20 min incubation period on a roller incubator, it was centrifuged for 30 min at 15000 rpm and the supernatant was taken off. The pellet was redispersed in 867 μl 2 mM MES buffer pH 6.5, and 133 μl MAB<Dig>M-IgG solution (monoclonal anti-digoxigenin IgG antibody from the mouse; concentration=15 mg/ml) was added. Afterwards it was incubated for 2 h at room temperature (RT). Subsequently 1 ml of a 2% solution of bovine plasma albumin (RPLA) in 5 mM potassium phosphate buffer pH 7.4 was added and it was incubated for a further 60 min at RT. The particle conjugates were centrifuged, the supernatant was removed and the pellet was resuspended in 1 ml 5 mM buffer. The potassium phosphate washing process was repeated twice and the particles were resuspended in 0.5 ml 2% RPLA in 5 mM Hepes buffer pH 7.4 after the last centrifugation step.

### EXAMPLE 5

### Use of <Dig> Silicate Particle Conjugates in a Strip Test

The test strips required to carry out the experiments consist of a plastic foil onto which an application fleece, a membrane and a suction fleece are glued. Two proteins, streptavidin and anti-mouse-IgG antibody, are immobilized on different lines on the membrane.

Poly-SA was immobilized on the target or result line i.e. the first line in the direction of chromatography and should specifically capture particles bound to digoxigenylated and biotinylated peptide by means of the biotin binding. The anti-mouse IgG antibodies were immobilized on the control line i.e. the second line in the direction of chromatography. These anti-mouse IgG antibodies should capture all excess particles that were not bound on the result line (conjugates of anti-digoxigenin antibodies from the mouse and the silicate particles).

Depending on the test strip variant the application fleece was impregnated with the sample material to be examined i.e. with 1 μg/ml or 0 μg/ml of a biotinylated and digoxigenylated peptide. A 100 mM Hepes buffer pH 7.5 (50 mM NaCl, 70 mM urea, 1 mM EDTA, 2% BPLA) was used to dilute the silicate particles and rewash the test strips. The silicate particles were diluted in Hepes buffer to a final concentration of 100 μg/ml.

Subsequently 60 μl of the described silicate particle dilution was pipetted on the reagent fleece and chromatographed for 10 min. Afterwards 40 μl Hepes buffer was pipetted onto the reagent fleece and chromatographed for a further 10 min. Finally the test strip was evaluated. Only the control line was visible in the absence of the peptide (=analyte) and in the presence of the peptide the result line was additionally visible.

The conjugates according to the invention of silicate particles coloured with halogen-containing dyes and biomolecules (in this case anti-digoxigenin antibodies) are thus suitable as detection reagents in an immunological test strip.

### LIST OF REFERENCES

Badley, R. D., et al., Langmuir 6 (1990) 792-801
Byers, C. H., et al., Ind. Eng. Chem. Res. 26 (1987) 1916-1923
Charles, R. G., und Roedel, E. P., J. Inorg. Nucl. Chem. 29 (1967) 715-723
EP 1 036 763
Fendler, J. H., Nanoparticles and Nanostructured Films, Wiley-VCH 1998, 180-183
Giesche, H., and Matijevic, E., Dyes and Pigments 17 (1991) 323-340
Giesche, H., J. European Ceramic Soc. 14 (1994) 189-204
Harris, T., et al., J. Non-Cryst. Solids 121 (1990) 307-403
Horn, D., and Rieger, J., Angew. Chem. 113 (2001) 4460-4492
Kawaguchi, H., Prog. Polym. Sci. 25 (2000) 1171-1210
Kron, J., et al., 2. Wörlitzer Workshop, Tagungsband 2000
Matijevic, et al., Dyes and Pigments 17 (1991) 323-340
Matsoukas, T., and Goulari, E., Colloid J. Interface Sci. 124 (1988) 252-261
Matsoukas, T., and Gulari, E., Colloid J. Interface Sci. 132 (1989) 13-21
Philipse, A. P., and Vrij, A., Colloid J. Interf. Sci. 12 (1989) 121-136
Quellet, et al., Colloid J. Interface Sci. 159 (1993) 150-7
Reisfeld, R., et al., J. of Alloys and Compounds 300-301 (2000), 147-151
Schwert, R., Dissertation, Würzburg (?)2000
Shibata, S., et al., J. Sol-Gel Sci. and Techn. 10 (1997) 263-268
Stöber, et al., Colloid J. Interface Sci. 26 (1968) 62-69
U.S. Pat. No. 5,102,763
U.S. Pat. No. 5,209,998
Van Blaaderen, A., and Vrij, A., Adv. Chem. Ser. 234 (1994) 83-111
Van Blaaderen, A., and Vrij, A., Colloid J. Interface Sci., 156 (1993) 1-18
Van Blaaderen, et al., Langmuir 8 (1992) 2921-2931
Van Helden, A. K., and Vrij, A., Colloid J. Interf. Sci. 81 (1981) 354-368
Verhaegh, and Van Blaaderen, A., Langmuir 10 (1994) 1427-1438
WO 93/10190

1. Sol-gel process for producing a metal oxide particle which contains at least one target molecule containing halogen in which, starting from known metal oxide precursors, the said precursor and the said target molecule are used, characterized in that a polyhalogenated metal alkylalkoxy compound is additionally used in the said sol-gel process.

2. Sol-gel process for producing a metal oxide particle containing at least one halogen-containing target molecule as claimed in claim 1, comprising the steps
a) production of a mixture containing the target molecule and a polyhalogenated metal alkylalkoxy compound,
b) starting the sol-gel process with the metal oxide precursor,
c) adding the solution from a),
d) optionally further addition of the metal oxide precursor and
e) ending the sol-gel process.

3. Process as claimed in claim 2, characterized in that 90 to 10% of the metal alkoxide precursor is used in step a) and 10 to 90% of the metal oxide precursor is used in step d).

4. Process as claimed in one of the claims 1 to 3, characterized in that based on the initial amount of metal oxide precursor between 0.04 and 0.4 mol % polyhalogenated metal alkylalkoxy compound is used.

5. Process as claimed in one of the claims 1 to 4, characterized in that based on the initial amount of metal oxide precursor between 0.1 and 10% by weight target molecule is used.

6. Process as claimed in one of the claims 1 to 5, characterized in that the halogen-containing target molecule is chlorinated or fluorinated.

7. Process as claimed in one of the claims 1 to 6, characterized in that the metal oxide is composed of B₂O₃, Al₂O₃, SiO₂, ZrO₂ or TiO₂ or mixed oxides thereof.

8. Metal oxide particle obtainable by the sol-gel process as claimed in one of the claims 1 to 7.

9. Use of a particle as claimed in claim 8 as a label for biomolecules.

10. Use of a particle as claimed in claim 8 as a sunscreen agent.

11. Use of a particle as claimed in claim 8 as a toner.

12. Use of a particle as claimed in claim 8 as an insecticide.
